(19) United States
(12) Patent Application Publication
(10) Pub. No.: US 2025/0354121 A1
(43) Pub. Date: Nov. 20, 2025

(54) DAIRY-BASED MICROSTRUCTURES AS MICROCARRIERS, SCAFFOLDS, SUBSTRATES, AND OTHER APPLICATIONS

(71) Applicant: Terasaki Institute for Biomedical Innovation, Los Angeles, CA (US)

(72) Inventors: Samad Ahadian, Los Angeles, CA (US); Wei Huang, Sherman Oaks, CA (US); Alireza Khademhosseini, Los Angeles, CA (US)

(73) Assignee: Terasaki Institute for Biomedical Innovation, Los Angeles, CA (US)

(21) Appl. No.: 18/876,797

(22) PCT Filed: Jun. 29, 2023
(86) PCT No.: PCT/US2023/026529

§ 371 Date: Dec. 19, 2024

Related U.S. Application Data
(60) Provisional application No. 63/358,374, filed on Jul. 5, 2022.
(60) Provisional application No. 63/358,346, filed on Jul. 5, 2022.

(51) Int. Cl. C12N 5/077; C12N 5/071; C12N 5/0735; C12N 5/074; C12N 5/0793

(52) U.S. Cl. CPC C12N 5/0658; C12N 5/0606; C12N 5/0619; C12N 5/0631; C12N 5/0653; C12N 5/0654; C12N 5/0656; C12N 5/066; C12N 5/069; C12N 5/0696; C12N 2501/998; C12N 2531/00; C12N 2537/10

(57) ABSTRACT

The present disclosure relates to dairy-based microstructures for cell culture and other applications. In some embodiments, the dairy-based microstructures comprise whey protein microparticles. In other embodiments, the microstructures comprise whey protein sheets. In certain cases, the microstructures comprise a coating, e.g., a protein, peptide, blood product, etc. In some embodiments, a plurality of cells may be cultured on the microstructures, e.g., animal cells. In other embodiments, the microstructures may be used to produce a cultivated product, e.g., a cell-based meat product. Other embodiments are generally directed to methods of making or using such microstructures for cell culture, kits involving these, or the like.

### RELATED APPLICATIONS

This application claims the benefit of U.S. Provisional Patent Application Ser. No. 63/358,374, filed Jul. 5, 2022, entitled “Cell Growth Supplements Using Dairy-Based Materials,” and U.S. Provisional Patent Application Ser. No. 63/358,346, filed Jul. 5, 2022, entitled “Dairy-Based Microstructures as Microcarriers, Scaffolds, Substrates, and Other Applications.” each of which is incorporated herein by reference in its entirety.

### FIELD

The present disclosure generally relates to dairy-based microstructures as microcarriers, scaffolds, and substrates for cell culture and other applications.

### BACKGROUND

Microcarriers are micron sized beads, e.g., microparticles, that support the attachment and growth of adherent cells in a cell culture system. In general, commercial microcarriers are engineered for specific applications and vary in chemical composition, charge, surface coatings, and porosity and allow cells to be cultured at a higher surface area per media volume than in planar culture. Currently, microcarriers are used for applications such as vaccine production, pharmaceutical production, and cell population expansion. However, one major disadvantage of existing commercial microcarriers, especially for cell expansion applications, is that the cells must be detached from the microcarrier surface for downstream applications. Improvements are therefore needed.

### SUMMARY

The present disclosure generally relates to dairy-based microstructures as microcarriers, scaffolds, and substrates for cell culture and other applications. The subject matter of the present disclosure involves, in some cases, interrelated products, alternative solutions to a particular problem, and/or a plurality of different uses of one or more systems and/or articles.

In some embodiments the present disclosure relates to a dairy-based microstructure comprising whey protein and a coating at least partially surrounding the whey protein. In other embodiments, the disclosure relates to a cultivated product comprising a plurality of dairy-based microstructures comprising a whey protein and a plurality of animal cells cultured on the microstructures.

Other embodiments are directed toward methods of use. For example, in some embodiments the method comprises culturing cells on a plurality of dairy-based microstructures comprising whey protein. In other cases, the method comprises culturing cells on a plurality of dairy-based microstructures, expanding the cells in a bioreactor by at least 10-fold, and dissolving the dairy-based microstructures to produce a cell suspension.

Some embodiments are directed toward methods of producing a dairy-based microstructure. For example, in some cases the method comprises dissolving whey protein in an aqueous solution to produce a whey protein solution and inducing the whey protein to form a plurality of microstructures.

In another aspect, the present disclosure encompasses methods of making one or more of the embodiments described herein, for example, a dairy-based microstructure. In still another aspect, the present disclosure encompasses methods of using one or more of the embodiments described herein, for example, a dairy-based microstructure.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which some of them are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates a process for preparing non-crosslinked and crosslinked whey microparticles, according to some embodiments;

FIG. 2A illustrates an optical photomicrograph of whey powder, according to some embodiments;

FIG. 2B illustrates an optical photomicrograph of crosslinked whey microparticles, according to some embodiments;

FIG. 2C illustrates an optical photomicrograph of non-crosslinked whey microparticles, according to some embodiments;

FIG. 2D is a comparative plot illustrating the difference in the mean area (microns\hat{ }2) of crosslinked whey microparticles, non-crosslinked whey microparticles, and whey powder, according to some embodiments;

FIG. 2E is a comparative plot illustrating the difference in the mean diameter (microns) of crosslinked whey microparticles, non-crosslinked whey microparticles, and whey powder, according to some embodiments;

FIG. 3A illustrates a fluorescent photomicrograph of primary bovine skeletal muscle cells cultured on a surface of non-crosslinked whey microparticles stained with a fluorescent live cell marker, calcein, according to some embodiments;

FIG. 3B illustrates a fluorescent photomicrograph of primary bovine skeletal muscle cells cultured on a surface of non-crosslinked whey microparticles stained with a fluorescent dead cell marker, ethidium homodimer, according to some embodiments;

FIG. 3C illustrates a brightfield optical photomicrograph of primary bovine skeletal muscle cells cultured on a surface of non-crosslinked whey microparticles, according to some embodiments;

FIG. 3D illustrates a fluorescent photomicrograph of primary bovine skeletal muscle cells cultured on a surface of crosslinked whey microparticles stained with a fluorescent live cell marker, calcein, according to some embodiments;

FIG. 3E illustrates a fluorescent photomicrograph of primary bovine skeletal muscle cells cultured on a surface of crosslinked whey microparticles stained with a fluorescent dead cell marker, ethidium homodimer, according to some embodiments;

FIG. 3F illustrates a brightfield optical photomicrograph of primary bovine skeletal muscle cells cultured on a surface of crosslinked whey microparticles, according to some embodiments;

FIG. 4A illustrates a fluorescent photomicrograph of primary bovine skeletal muscle cells cultured on a surface of a single non-crosslinked whey microparticle stained with a fluorescent live cell marker, calcein, according to some embodiments;

FIG. 4B illustrates a fluorescent photomicrograph of primary bovine skeletal muscle cells cultured on a surface of a single non-crosslinked whey microparticle stained with a fluorescent dead cell marker, ethidium homodimer, according to some embodiments;

FIG. 4C illustrates a brightfield optical photomicrograph of primary bovine skeletal muscle cells cultured on a surface of a single non-crosslinked whey microparticle, according to some embodiments;

FIG. 4D illustrates a fluorescent photomicrograph of primary bovine skeletal muscle cells cultured on a surface of a single crosslinked whey microparticle stained with a fluorescent live cell marker, calcein, according to some embodiments;

FIG. 4E illustrates a fluorescent photomicrograph of primary bovine skeletal muscle cells cultured on a surface of a single crosslinked whey microparticle stained with a fluorescent dead cell marker, ethidium homodimer, according to some embodiments;

FIG. 4F illustrates a brightfield optical photomicrograph of primary bovine skeletal muscle cells cultured on a surface of a single crosslinked whey microparticle, according to some embodiments;

FIG. 5A is a comparative plot illustrating the difference in the cell viability (%) of primary bovine skeletal muscle cells cultured on crosslinked whey microparticles versus non-crosslinked whey microparticles, according to some embodiments;

FIG. 5B is a comparative plot illustrating the difference in the absolute cell number (per 50 mL of cell suspension) of primary bovine skeletal muscle cells cultured on crosslinked whey microparticles versus non-crosslinked whey microparticles, according to some embodiments;

### DETAILED DESCRIPTION

The present disclosure relates to dairy-based microstructures for cell culture and other applications. In some embodiments, the dairy-based microstructures comprise whey protein microparticles. In other embodiments, the microstructures comprise whey protein sheets. In certain cases, the microstructures comprise a coating, e.g., a protein, peptide, blood product, etc. In some embodiments, a plurality of cells may be cultured on the microstructures, e.g., animal cells. In other embodiments, the microstructures may be used to produce a cultivated product, e.g., a cell-based meat product. Other embodiments are generally directed to methods of making or using such microstructures for cell culture, kits involving these, or the like.

Certain aspects of the disclosure generally relate to dairy-based microstructures for cell culture and other applications. In some embodiments, the dairy-based microstructures comprise a milk protein, e.g., whey protein or casein protein, etc. As discussed herein, milk proteins, e.g., whey protein, may be processed into any microstructure, e.g., microparticles, sheets, etc. The whey proteins, in some embodiments, may be crosslinked or non-crosslinked. In some embodiments, a binding agent, e.g., transglutaminase, may be used to at least partially crosslink the whey protein microstructures.

In some embodiments, the dairy-based microstructures comprising whey protein, e.g., microparticles, may comprise a coating that at least partially surrounds the whey protein. In some embodiments, the coating may be covalently and/or non-covalently coupled to the dairy-based microstructure. The coating may comprise components such as cell-binding ligands, e.g., RGDS; extracellular matrix products, e.g., laminin; blood serum products, e.g., fibrinogen; etc.

In some embodiments, the dairy-based microstructures, e.g., whey protein microparticles, may be used to expand a culture of animal cells, e.g., muscle cells, nerve cells, fat cells, etc. In some embodiments, the cells may comprise stem cells, such as embryonic stem cells, induced pluripotent stem cells, adult stem cells, and the like. In certain cases, the animal cells may arise from any appropriate source, including, human, cow or bovine, sheep or ewe, pig or swine, camel, horse, crocodile, bison, and the like. In some cases, the cells may be cultured on the dairy-based microstructures, and in certain instances, expanded to at least 2×, at least 4×, at least 10×, etc., the initial cell number. In certain embodiments, cells may be cultured on the microstructures to produce a cultivated product, e.g., skin, leather, wool, organs, etc. Other applications are also possible. For example, in some embodiments, dairy-based microstructures may be used to study cell biology, e.g., the effect of substrate stiffness on various differentiation pathways in stem cells.

Some aspects of the disclosure are directed to methods for procuring milk products, e.g., whey and casein protein, for producing dairy-based microstructures, for example, for cell culture. In some cases, the methods comprise obtaining milk from a milk-producing animal, such as a cow or goat. In some embodiments, the milk-producing animal is a pregnant and/or a nursing animal. In certain embodiments, the milk-producing animal may be artificially induced to produce milk without being a pregnant and/or nursing animal. In certain cases, one or more milk proteins may be isolated from the obtained milk product, e.g., whey protein, and fabricated into a dairy-based microstructure, e.g., a microparticle and/or 2D sheet. In some cases, a plurality of cells, e.g., animal cells, may be cultured on the microstructure to yield a product, e.g., a cultivated product.

Some aspects of the disclosure relate to compositions of dairy-based microstructures and methods of making or using such microstructures, e.g., microcarriers, scaffolds, substrates, etc., for cell culture and other applications. In some embodiments, the dairy-based microstructures comprise a milk protein, e.g., whey protein or casein protein. In some cases, the whey protein may be purchased from a commercial vendor, such as GNC, Glanbia Nutritionals, Superior Supplement Manufacturing, Makers Nutrition, NutraScience Labs, etc. In addition, in certain embodiments, the whey protein may be extracted, by those skilled in the art, directly from a milk product, e.g., such as milk obtained directly from a milk-producing animal, according to some embodiments. In addition, in certain cases, the whey protein may be harvested from, for example, a bacterial culture.

In some cases, the dairy-based microstructures comprise a plurality of microparticles, e.g., comprising whey protein. In some embodiments, the plurality of microparticles comprise whey proteins that are crosslinked using, for example, a crosslinking agent, e.g., transglutaminase, glutaraldehyde, DIDS, 1,14-Diazido-3,6,9,12-tetraoxatetradecane, 1,1,-methanediyl bismethanethiosulfonate, etc. However, in some cases, the plurality of microparticles comprise whey proteins that are not crosslinked.

In some embodiments, the plurality of microparticles may have a mean diameter of greater than or equal to 10 microns, greater than or equal to 20 microns, of greater than or equal to 30 microns, greater than or equal to 40 microns, greater than or equal to 50 microns, greater than or equal to 60 microns, etc. In some embodiments, the plurality of microparticles may have a mean diameters of less than or equal to 60 microns, of less than or equal to 50 microns, of less than or equal to 40 microns, less than or equal to 30 microns, less than or equal to 20 microns, less than or equal to 10 microns, etc. In some embodiments, the plurality of microparticles may have a mean area of greater than or equal to 50 microns², greater than or equal to 100 microns², greater than or equal to 200 microns², greater than or equal to 300 microns², greater than or equal to 400 microns², greater than or equal to 500 microns², greater than or equal to 600 microns², etc. In some embodiments, the plurality of microparticles may have a mean area of less than or equal to 600 microns², of less than or equal to 500 microns², of less than or equal to 400 microns², of less than or equal to 300 microns², of less than or equal to 200 microns², of less than or equal to 100 microns², of less than or equal to 50 microns², etc. In some embodiments, the plurality of microparticles may have an aspect ratio of greater than or equal to 0.1:1, greater than or equal to 0.5:1, of greater than or equal to 0.75:1 microns, of greater than or equal to 1:0.75, of greater than or equal to 1:0.5, of greater than or equal to 1:0.1, etc. In some embodiments, the plurality of microparticles may have an aspect ratio of less than or equal to 1:0.1, of less than or equal to 1:0.5, of less than or equal to 1:0.75, of less than or equal to 0.75:1, of less than or equal to 0.5:1, of less than or equal to 0.1:1, etc.

In some embodiments, the dairy-based microstructures comprise a plurality of sheets, e.g., comprising whey protein. In some embodiments, the plurality of sheets comprises whey proteins that are crosslinked using, for example, a crosslinking agent, e.g., transglutaminase, glutaraldehyde, DIDS, 1,14-Diazido-3,6,9,12-tetraoxatetradecane, 1,1,-methanediyl bismethanethiosulfonate, etc. However, in some embodiments, the plurality of sheets comprises whey proteins that are not crosslinked.

In some embodiments, the plurality of sheets may have a mean square surface area of greater than or equal to 100 microns², greater than or equal to 500 microns², of greater than or equal to 1000 microns², of greater than or equal to 2500 microns², etc. In some embodiments, the plurality of sheets may have a mean square surface area of less than or equal to 2500 microns², of less than or equal to 1000 microns², of less than or equal to 500 microns², of less than or equal to 100 microns², etc. In some embodiments, the plurality of sheets may have a mean thickness of greater than or equal to 1 micron, greater than or equal to 10 microns, of greater than or equal to 50 microns, of greater than or equal to 100 microns, of greater than or equal to 1000 microns, etc. In some embodiments, the plurality of sheets may have a mean thickness of less than or equal to 1000 microns, of less than or equal to 100 microns, of less than or equal to 50 microns, of less than or equal to 10 microns, of less than or equal to 1 micron, etc. In some embodiments, the plurality of sheets may have an aspect ratio of greater than or equal to 0.1:1, greater than or equal to 0.5:1, of greater than or equal to 0.75:1 microns, of greater than or equal to 1:0.75, of greater than or equal to 1:0.5, of greater than or equal to 1:0.1, etc. In some embodiments, the plurality of sheets may have an aspect ratio of less than or equal to 1:0.1, of less than or equal to 1:0.5, of less than or equal to 1:0.75, of less than or equal to 0.75:1, of less than or equal to 0.5:1, of less than or equal to 0.1:1, etc.

In some embodiments, a dairy-based microstructure may also include a coating that at least partially surrounds a whey protein. For example, the microstructure may comprise microparticles, sheets, etc., that are partially or fully coated with a coating. The coating may, for example, promote cellular adhesion and/or proliferation, provide attachment for additional functionalities (e.g., a surface to attach proteins), protect the protein, or other applications.

In some embodiments, for example, the coating may comprise a cell-binding ligand, e.g., a cell-adhesion peptide such as RGDS, RGD, PHSRN, PA22-2 (CSRARKQAASIKVAVSADR-NH₂), RU-1 (LNIVSVNGRHX), RX-1 (DNRIRLQAKXX), GD-1 (KATPMLKMRTSFHGCIK), GD-2 (KEGYKVRLDLNITLEFRTTSK), GD-3 (KNLEISRSTFDLLRNSYGVRK), GD-6 (KQNCLSSRASFRGCVRNLRLSR), HGD-6 (KQKCLRSQTSFRGCLRKLALIK), SGD-6 (CRNRGRCNSSLFQVRSRKLLSA), HSGD-6 (KQCLKSQRSFTRGLCRLKAKIL), AG-1 (KLLISRARKQÁASIK), F17 (LERKYENDQKYLEDKA) and KRGD (VEKRGDREEA).

In another set of embodiments, the coating may comprise at least one component of an extracellular matrix. Examples of such components of an extracellular matrix include, but are not limited to proteoglycans (e.g., heparan sulfate, chondroitin sulfate, keratan sulfate, etc.), non-proteoglycan polysaccharides (e.g., hyaluronic acid), proteins (e.g., collagen, elastin, fibronectin, laminin, etc.), a blood serum product (e.g., proteins such as albumin, globulin, immunoglobin, prothrombin, fibrinogen, electrolytes, antibodies, antigens, hormones, and the like). Combinations of any of these and/or other components are also possible, e.g., the coating may comprise at least one extracellular matrix component and at least one blood serum component.

In some embodiments, the coating may comprise specific binding elements, such as biotin, avidin, streptavidin, neutravidin, lectins, SNAP-tags™ or substrates therefore, associative or binding peptides or proteins, antibodies or antibody fragments, nucleic acids or nucleic acid analogs, or the like. Additionally, or alternatively, the coating may be used to couple an additional binding element that is used to couple or bind to a specific article of interest, e.g., a substrate, which may, in some cases include both chemical groups and/or specific binding elements. By way of example, a binding element, e.g., biotin, may be deposited upon a dairy-based microstructure, e.g., a microparticle. An intermediate binding agent, e.g., streptavidin, may then be coupled to the biotin binding element. A compound of interest, e.g., a biotinylated antibody, may then be coupled to the streptavidin, thus immobilizing the antibody to the microstructure.

In some embodiments, a coating may be covalently coupled to an outer surface of a dairy-based microstructure. Without wishing to be bound by theory, whey protein comprises amino acids with reactive side groups that may be used to covalently couple at least a portion of the whey protein to the coating. For example, whey protein comprises about 8% aspartic acid and about 13% glutamic acid, each of which comprise a reactive carboxyl group, about 8% lysine, which comprises a reactive primary amine, and about 2% cystine, which comprises a reactive sulfhydryl group, among others. The carboxyl group of aspartic acid and glutamic acid is a strong electrophile capable of reacting with nucleophilic compounds (e.g., compounds containing free amines (—NH₂), free sulfhydryl groups (—SH), free hydroxide groups (—OH), carboxylates, hydrazides, and alkoxyamines). Similarly, the primary amine and sulfhydryl groups of lysine and cystine, respectively, are strong nucleophiles capable of reacting with electrophilic compounds (e.g., compounds containing maleimides, N-hydroxysuccinimide (NHS) activated esters, carbodiimides, hydrazides, tetrafluorophenyl (TFP) esters, pentafluorophenyl (PFP) esters, phosphines, hydroxymethyl phosphines, psoralen, imidoesters, pyridyl disulfides, isocyanates, vinyl sulfones, alpha-haloacetyls, aryl azides, acyl azides, alkyl azides, diazirines, benzophenone, epoxides, carbonates, anhydrides, sulfonyl chlorides, cyclooctynes, and aldehydes).

For example, in some embodiments, a coating comprising RGDS may be covalently coupled to the outer surface of a dairy-based microstructure, e.g., a microparticle, by treating the microstructure with an RGDS compound bearing, for example, a pentafluorophenyl ester, e.g., Ac-RGDS-OPfp. In this exemplary embodiment, the RGDS peptide contains an activated ester that is susceptible to nucleophilic attack by, for example, the side chain lysine (i.e., free amine group), thus linking the RGDS coating to the microstructure surface via an amide bond.

In some embodiments, a coating may be non-covalently coupled to an outer surface of a dairy-based microstructure. Without wishing to be bound by theory, it is believed that whey protein comprises a plurality of amino acids with ionizable side groups, e.g., lysine, aspartic acid, glutamic acid, etc., that may permit modulation of the surface zeta potential. For instance, the isoelectric point (pI) for whey protein is approximately 5.5, and when the pH of a whey protein solution is greater than its pI, the solubilized whey proteins are predominately negatively charged and when the pH of the whey protein solution is less than its pI, the solubilized whey proteins are predominately positively charged. In some embodiments, the non-covalent coating comprises depositing alternating layers of oppositely charged materials (e.g., cationic and/or anionic) onto the outer surface of the dairy-based microstructure. Exemplary embodiments of charged materials include proteins, e.g., blood serum proteins and/or extracellular matrix proteins, antibodies, etc., nucleic acids, e.g., polynucleic acids, oligonucleotides, etc., polymers, e.g., poly(ethylenimine), poly(2-dimethyl(aminoethyl) methacrylate), poly-L-lysine, polyglutamic acid, polyaspartic acid, chitosan, etc., carbohydrates, e.g., gelatin, dextran sulfate, alginate, hyaluronic acid, etc., or the like.

In some embodiments, the coating may comprise greater than 2 layers, greater than 3 layers, greater than 4 layers, greater than 5 layers, greater than 6 layers, greater than 7 layers, greater than 8 layers, greater than 9 layers, greater than 10 layers, etc. In some embodiments, the coating may comprise less than 10 layers, less than 9 layers, less than 8 layers, less than 7 layers, less than 6 layers, less than 5 layers, less than 4 layers, less than 3 layers, less than 2 layers, etc. In certain embodiments, the zeta potential (i.e., surface charge) of the outer surface of the microstructure may be varied from highly negative (e.g., less than 1) to highly positive (e.g., greater than 1), depending on the chemical composition of the terminal coating (e.g., terminating with poly-L-lysine may create a positive zeta potential, the magnitude of which, may depend on the total number of layers in some cases).

In some embodiments, a dairy-based microstructure may be placed into a cell culture medium comprising at least one component of a blood serum, for example, to aid in the growth of cells. In some cases, compounds in the cell culture medium may adsorb onto the charged outer surface of the dairy-based microstructure via electrostatic interactions, hydrogen bonding, halogen bonding, van der Waals forces (e.g., dipole-dipole interactions, dipole-induced dipole interactions, London dispersion forces), pi effects, and/or hydrophobic effects. For example, positively charged materials, e.g., proteins, polysaccharides, etc., within the cell culture medium may bind to the whey protein-based microstructure with the negatively charged outer surface, thus forming a positively charged coating that is non-covalently coupled to the outer surface of the microstructure. Similarly, negatively charged materials, e.g., fibrinogen, albumin, immunoglobin, antibodies, coagulation factors, globins, etc., within the cell culture medium may bind to the whey protein-based microstructures with a positively charged outer surface, thus forming a negatively charged coating that is non-covalently coupled to the outer surface of the microstructure. In certain cases, microstructures with a plurality of multi-layered coatings that are non-covalently coupled to the outer surface of the microstructure may be produced. In some embodiments, a first portion of the microstructures may comprise a terminal coating with a positive charge, while a second portion of the microstructures may comprise a negative charge. In certain embodiments, microstructures with positively and/or negatively charged coatings on their outer surfaces may enhance cellular adhesion to the microstructure and/or enhance cell proliferation of adhered cells.

In some embodiments, a dairy-based microstructure may comprise a first coating covalently coupled to the outer surface. In some embodiments, there may be a first coating that may comprise a plurality of ionizable groups, such as a carboxyl group or a primary amino group, thus permitting deposition of a second coating, wherein the second coating comprises alternating layers of oppositely charged materials (e.g., cationic and/or anionic). In some embodiments, the first coating is non-covalently coupled to the outer surface and comprises a plurality of reactive ionizable groups, such as the carboxyl group, the primary amino group, etc. In certain cases, the reactive ionizable groups may be covalently coupled to the second coating, where the second coating comprises materials with the appropriate complementary reactive group. For instance, in one exemplary embodiment, a first coating comprising a plurality of primary amines may be covalently conjugated to a protein, e.g., an antibody, comprising at least one activated ester, e.g., NHS, OPfp, etc.

In some embodiments, a dairy-based microstructure may be used in a culture of animal cells. For example, in some embodiments, a plurality of animal cells may be cultured on the plurality of microparticles and/or plurality of sheets. In some embodiments, the plurality of animal cells may include cells such as myoblasts, fibroblasts, adipocytes, vascular cells, osteoblasts, tenocytes, epithelial cells, mammalian glands, neural cells, etc. In some cases, the cells may include stem cells, such as embryonic stem cells, induced pluripotent stem cells, adult stem cells, etc. In certain embodiments, the animal cells may arise from any source, including mammals (e.g., humans, cats, dogs, horses, duckbill platypuses, kangaroos, dolphins, whales, etc.), birds, fish, reptiles (e.g., lizards, crocodiles, alligators, turtles, etc.), amphibians, and arthropods, etc. In certain cases, the microstructures may be used to produce one or more cultivated products, for example, a cell-based meat, horn, leather, skin, wool, organ, milk, etc.

Some aspects of the disclosure relate to a cultivated product, wherein the cultivated product comprises a plurality of dairy-based microstructures, e.g., microparticles, comprising whey protein. In some embodiments, a plurality of animal cells may also be cultured on the microparticles. In some embodiments, microstructures may comprise a coating, e.g., laminin, fibrinogen, etc. The coating may, for example, enhance adhesion of animal cells to the microstructures, and/or enhance their proliferation. In some embodiments, the coating may comprise a protein, a peptide, a nucleic acid, polynucleic acid, oligonucleotide, polysaccharide, carbohydrate, etc., e.g., such as described herein.

In certain embodiments, the coating may comprise at least one component of a blood product, for example, fibrinogen. In some instances, the coating may comprise at least one component of an extracellular matrix, such as a laminin, collagen, or hyaluronic acid. The coating may be covalently coupled in some cases to the microstructure surface. In some embodiments, the coating may be non-covalently coupled to the microstructure surface, for example, a microparticle surface. In certain cases, multiple coatings may be coupled, either covalently and/or non-covalently, to the microstructure surface. In some embodiments, the cultivated product may be an edible product, such as a cell-based meat product or milk. In other embodiments, the cultivated product may be a textile, such as for example, wool, rhino horn, elephant tusks, leather, etc. In some embodiments, the cultivated product may be a medical product, such as skin or a cell-based organ for transplantation.

Some aspects of the disclosure relate to culturing cells on a dairy-based microstructures, e.g., microparticles and/or sheets, comprising whey protein. For example, in some embodiments, dairy-based microstructures, e.g., microcarriers, can be added to a bioreactor, or other cell culture system. The bioreactor or other cell culture system may contain cells, e.g., suspended in a cell culture growth media comprising a serum product, whey protein, fetal bovine serum, blood product, or the like. The cells may be cultured with the microstructures. Blood products may include, e.g., plasma, platelet-rich plasma, platelet-poor plasma, serum, platelet lysate, etc. In some embodiments, culturing the cells produces a cultivated product.

In some embodiments, a plurality of cells may be cultured on microstructures until the microstructures dissolve. Dissolving the microstructures, according to some embodiments, dissociates the plurality of cells from the plurality of microstructures, e.g., into a single cell suspension. For instance, in some cases, the cells may release an enzyme that degrades the microstructure. Examples include proteases, e.g., an aspartic proteases, glutamic protease, metalloproteases, cysteine protease, serine protease, threonine protease, etc.

In some embodiments, the plurality of cells may be cultured on the microstructures for greater than or equal to 2 days, greater than or equal to 4 days, greater than or equal to 6 days, greater than or equal to 8 days, greater than or equal to 10 days, greater than or equal to 20 days, greater than or equal to 30 days, etc. In some embodiments, the plurality of cells may be cultured on the microstructures for less than or equal to 30 days, for less than or equal to 20 days, for less than or equal to 10 days, for less than or equal to 8 days, for less than or equal to 6 days, for less than or equal to 4 days, for less than or equal to 2 days, etc. Combinations of these are also possible in certain cases, e.g., the cells may be cultured for between 2 days and 8 days, between 10 days and 30 days, etc.

In some embodiments, dairy-based microstructures comprising whey protein may be prepared using a water-in-oil emulsion technique. The whey protein may be purchased by commercial vendors or isolated from a milk product by those of ordinary skill in the art. In some embodiments, whey protein is dissolved in phosphate buffered saline to produce a whey protein solution. The whey protein solution may be stirred for at least 1 hour, at least 2 hours, at least 3 hours, at least 4 hours, at least 5 hours, at least 10 hours, at least 15 hours, at least 24 hours, etc., to begin hydration of the whey protein. The hydrated whey protein solution may be filtered, e.g., using a 0.45-micron filter, and the solution stored at a temperature of at least 4° C., at least 25° C., at least 37° C., etc., for at least 12 hours, at least 24 hours, at least 36 hours, at least 48 hours, at least 72 hours, etc., to hydrate the whey protein.

In some embodiments, the hydrated whey protein solution may be homogenized in an oil solution, e.g., comprising at least one surfactant or emulsifier to produce a water-in-oil (w/o) emulsion comprising whey protein microdroplets. A variety of oil phases, e.g., nonane, decane, undecane, n-dodecane, etc., and emulsifiers, e.g., sorbitan, stearate, polyglycerol oleate, lecithin, sorbitan monooleate, lanolin, olyglycerolpolyricinoleate, etc., may be used to produce the w/o emulsion. In some cases, the ratio of whey protein solution to emulsifier solution is greater than or equal to 1:4, greater than or equal to 1:5, greater than or equal to 1:6, greater than or equal to 1:7, greater than or equal to 1:8, greater than or equal to 1:9, greater than or equal to 1:10, greater than or equal to 1:12. In some embodiments, the ratio of whey protein solution to emulsifier solution is less than or equal to 1:12, less than or equal to 1:10, less than or equal to 1:9, less than or equal to 1:8, less than or equal to 1:7, less than or equal to 1:6: less than or equal to 1:5, less than or equal to 1:4, etc. In certain cases, the solution may be homogenized at a speed ranging between 7,000 rpm and 25,000 rpm. Following homogenization, the w/o emulsion may be heated to heat-gel the whey protein microdroplets to form stable whey protein microparticles, and then cooling the solution. In some embodiments, the method comprises heating the solution at least 50° C., at least 60° C., at least 70° C., at least 80° C., at least 90° C., at least 100° C., etc., for at least 5 minutes, at least 10 minutes, at least 15 minutes, at least 20 minutes, at least 30 minutes, at least 45 minutes, at least 60 minutes, etc. In some embodiments, the microparticles may be treated them with a crosslinking agent, such as transglutaminase, to crosslink the whey proteins within the microparticle.

In certain embodiments, fabricating the dairy-based microstructures, e.g., microparticles, comprising whey protein uses a process known as salt-induced precipitation. Salt-induced precipitation (also known as salting out, salt fractionation, antisolvent crystallization, precipitation crystallization, or drowning out) is a technique that utilizes the reduced solubility of certain molecules in a solution of very high ionic strength. In some embodiments, the whey protein is hydrated in an aqueous solution, e.g., phosphate buffered saline to produce a hydrated whey protein solution. Following hydration, according to some embodiments, the whey protein solution may be heated to a temperature of at least 50° C., at least 60° C., at least 70° C., at least 80° C., at least 90° C., at least 100° C., etc., for at least 5 minutes, at least 10 minutes, at least 15 minutes, at least 20 minutes, at least 30 minutes, at least 45 minutes, at least 60 minutes, etc. Thereafter, the pH of the whey protein solution may be adjusted to a pH of at least 6.0, of at least 6.5, of at least 7.0, of at least 7.5, of at least 8.0, of at least 8.5, etc.

In some cases, a sodium chloride solution can be added to the whey protein solution, thus inducing the precipitation of microstructures. In some embodiments, the sodium chloride solution may have a concentration in the range of between 50 microM and 2.5M. For example, in some embodiments, the sodium chloride concentration may be greater than or equal to 50 microM, greater than or equal to 100 microM, greater than or equal to 1 mM, greater than or equal to 1M, greater than or equal to 2M, greater than or equal to 2.5M, etc. In some embodiments, the sodium chloride concentration less than or equal to 2.5M, less than or equal to 2 M, less than or equal to 1 M, less than or equal to 1 mM, less than or equal to 100 microM, less than or equal to 50 microM, etc.

In certain cases, a crosslinking agent, e.g., transglutaminase, can be added to the hydrated whey protein solution, thus crosslinking the dissolved whey proteins and inducing their assembly into nanoaggregates. Any crosslinking agent may be used to crosslink the protein-based microstructures, e.g., transglutaminase, disuccinimidyl suberate, sulfosuccinimidyl-4-(N-maleimidomethyl) cyclohexane-1-carboxylate, carbodiimides, etc. In some embodiments, the sodium chloride solution is added to the crosslinked solution, thus inducing the assembly of the whey protein nanoaggregates into microparticles. The sodium chloride may be present in the crosslinked solution may have a concentration in the range of between 50 microM and 2.5 M. For example, in some embodiments, the sodium chloride concentration may be greater than or equal to 50 microM, greater than or equal to 100 microM, greater than or equal to 1 mM, greater than or equal to 1 M, greater than or equal to 2 M, greater than or equal to 2.5 M, etc. In some embodiments, the sodium chloride may be present in the crosslinked solution at less than or equal to 2.5 M, less than or equal to 2 M. less than or equal to 1 M, less than or equal to 1 mM, less than or equal to 100 microM, less than or equal to 50 microM, etc.

Some embodiments are generally directed to obtaining milk from a milk-producing animal, extracting a whey protein from the milk, and using the whey protein to fabricate a plurality of microparticles for cell culture. Obtaining the milk product, may include performing milkings on an animal that is actively nursing, e.g., an animal that has recently given birth, or on an animal that is pregnant and is within at least 1 month, at least 2 months, at least 3 months, at least 4 months, at least 5 months, at least 6 months, at least 7 months, at least 8 months, at least 9 months, at least 10 months of giving birth. In some embodiments, the animals are continually impregnated, e.g., using artificial insemination, so that they lactate for up to 10 months. In some cases, obtaining milk from the milk producing animal comprises performing a plurality of milkings on an animal artificially induced to lactate. Methods of artificially inducing lactation in animals, e.g., heifers, are generally known to those of ordinary skill in the art, and may involve administering a series of injections comprising various lactation inducing drugs, e.g., hormones such as estrogen, progesterone, insulin, growth hormone, cortisol, thyroxine, human placental lactogen, etc. For example, in some embodiments, high doses of oestrogen plus progesterone may be administered over prolonged periods to develop the animals' mammary glands, followed by relatively high dose of estrogen to initiate milk secretion. In other embodiments, repeated injections of dexamethasone trimethylacetate may be administered following mammary gland development, for example, to enhance milk production.

In some embodiments, performing a plurality of milkings on an animal, e.g., a cow, capable of producing milk, produces a volume of between 9 liters and 13 liters of milk per kg of weight of the milk producing animal over a 10-month period. For example, a dairy cow with a body weight ranging from 600 kg to 900 kg may produce an average of 28 liters of milk per day over a period of 10-month period. In some embodiments, performing a plurality of milkings produces between 50 and 200 milligrams of whey protein per mL of milk product.

U.S. Provisional Patent Application Ser. No. 63/358,374, filed Jul. 5, 2022, entitled “Cell Growth Supplements Using Dairy-Based Materials,” and U.S. Provisional Patent Application Ser. No. 63/358,346, filed Jul. 5, 2022, entitled “Dairy-Based Microstructures as Microcarriers, Scaffolds, Substrates, and Other Applications,” each are incorporated herein by reference in its entirety.

The following examples are intended to illustrate certain embodiments of the present disclosure, but do not exemplify the full scope of the disclosure.

### Example 1

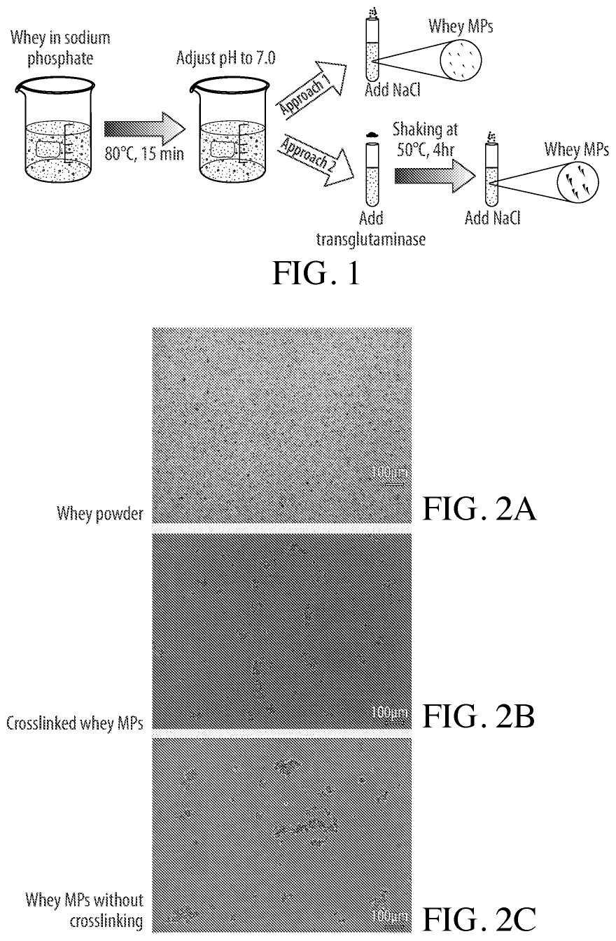

Preparation of whey microparticles. Whey microparticles were prepared in this example as either non-crosslinked whey microparticles or crosslinked whey microparticles using one of two protocols (FIG. 1). For both protocols, a commercial whey protein was used as the source of protein, however, as described elsewhere herein, the whey protein may also be isolated from a milk product, for example, from a cow or goat.

Protocol 1: Whey protein was dissolved in phosphate buffered saline at a final concentration of 150 g per kg of protein. The solution was subsequently stirred for 2 hours, filtered using a 0.45-micron hydrophilic syringe filter, and left at 4° C. overnight to complete hydration of the whey protein. Separately, a solution of polyglycerolpolyricinoleate (PGPR) was prepared, in n-dodecane at a final concentration of 25 g per kg and stirred for 1 hour at 40° C. The whey protein solution (2 g) was then added to the PGPR solution (18 g), and the water-in-oil (w/o) emulsion homogenized using a high-speed mixer for approximately 5 minutes. The size of the whey protein droplet (i.e., the water droplet) was varied by varying the emulsion speed between 7,000 rpm and 25,000 rpm. Following homogenization, the w/o emulsion was dispensed into sealed glass tubes and heated to 80° C. for 15 minutes, in order to heat-gel the microdroplets to form stable microparticles, and then rapidly cooled in ice water until they reached room temperature. The microparticles were separated from the w/o emulsion and repeatedly rinsed to remove all non-gelled material. The microparticles were then redispersed in phosphate buffered and split into two batches. A first batch was used as is, whereas a second batch was treated with transglutaminase to crosslink the microparticles.

Protocol 2: Whey protein was dissolved in phosphate buffered saline at a final concentration of 150 g per kg of protein. The solution was subsequently stirred for 2 hours, filtered using a 0.45-micron hydrophilic syringe filter, and left at 4° C. overnight to complete hydration of the whey protein. Following hydration, the whey protein was heated to 80° C. for 15 mins and the pH was adjusted to 7.0. Non-crosslinked whey microparticles were produced by addition a sodium chloride solution (100 microM) to the whey solution, thus salting out the dissolved whey as whey microparticles. Crosslinked whey microparticles were produced by addition of transglutaminase (100 microM) to the whey solution, thus crosslinking the dissolved whey into whey microparticles. The microparticles were separated from whey solution and repeatedly rinsed. The microparticles were then redispersed in phosphate buffered saline and stored until use.

### Example 2

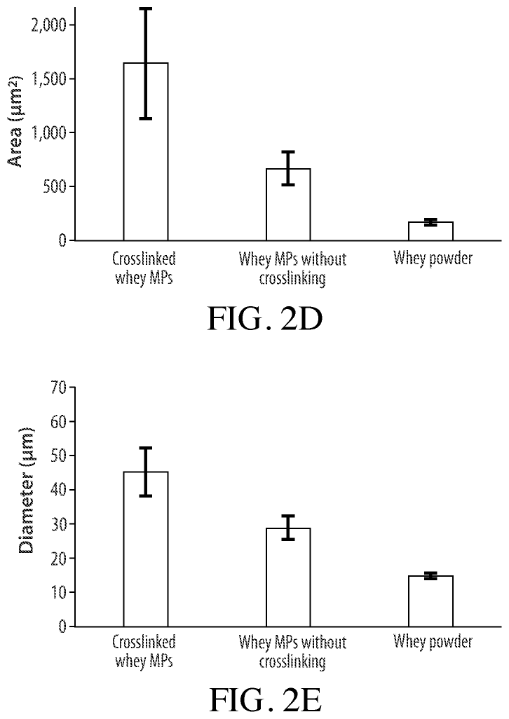

Whey protein, crosslinked whey microparticles, and non-crosslinked whey microparticles were characterized in this example to determine their mean area and mean diameter. Solutions of whey powder, crosslinked whey microparticles, non-crosslinked whey microparticles were prepared as described in Example 1. An aliquot was dispersed on a glass microscope, and optical photomicrographs taken (FIGS. 2A-2C). The images were subsequently processed using imaging software (IMAGEJ) to determine the mean area (square microns) and mean diameter of the microparticles. The results indicated that crosslinked whey microparticles had a consistently larger mean area and mean dimeter, compared to non-crosslinked microparticles. This is believed to be because the crosslinked whey microparticles can absorb water, which causes them to swell, whereas the non-crosslinked microparticles cannot absorb water.

### Example 3

The potential for crosslinked whey microparticles and non-crosslinked whey microparticles to support cell growth (e.g., as cell microcarriers) was tested in this example using primary bovine skeletal muscle cells. The cells were loaded into well plates at a density of 5×10⁶ cells/per 50 mL. To this was added either 0.1 g of a 5% (w/w) solution of microparticles comprising crosslinked whey protein or 0.1 g of a 5% (w/w) solution of microparticles comprising non-crosslinked whey proteins. All solutions were prepared using DMEM basal media supplemented with 10 (v/v) % FBS, and 1 (v/v) % penicillin-streptomycin. The well plates were placed in a humidified incubator in 5% CO₂ and 37° C. and left for up to 8 days to allow time for cell attachment and proliferation. After 3 or 8 days, a live/dead assay was performed to determine the viability of the cells and to determine the degree of proliferation. Cells were stained with a cocktail comprising calcein (live stain) and ethidium homodimer (dead stain).

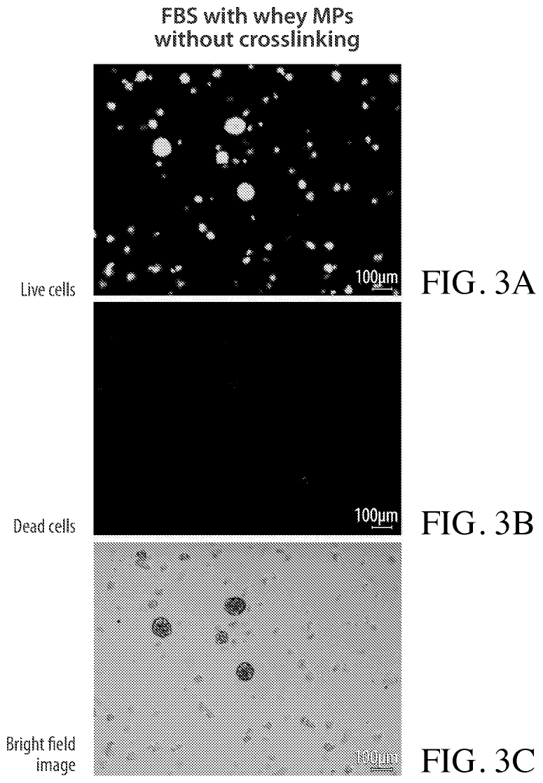

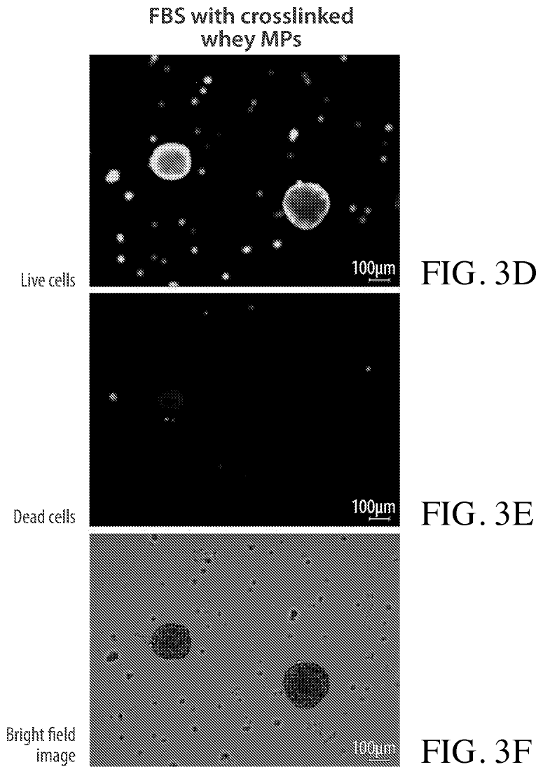

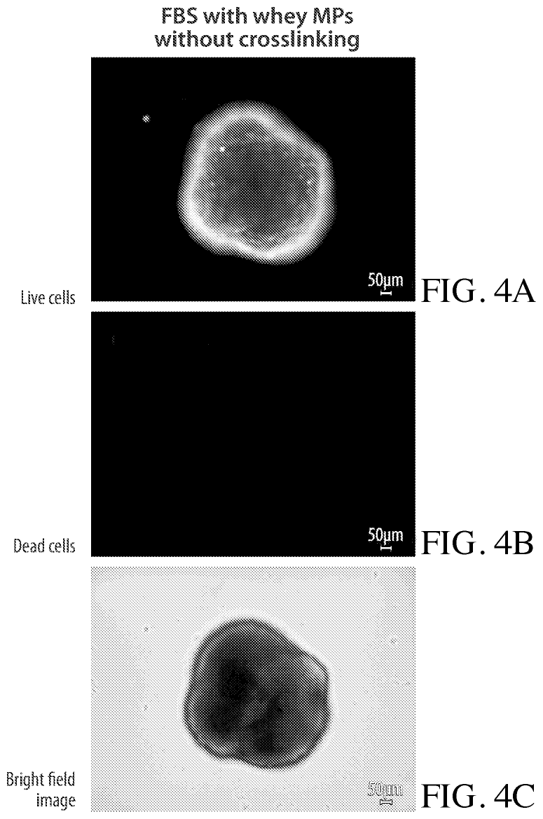

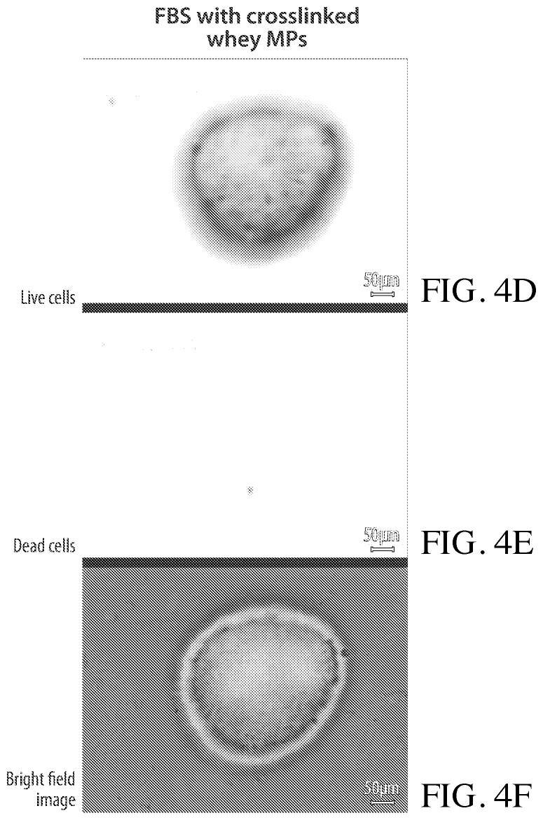

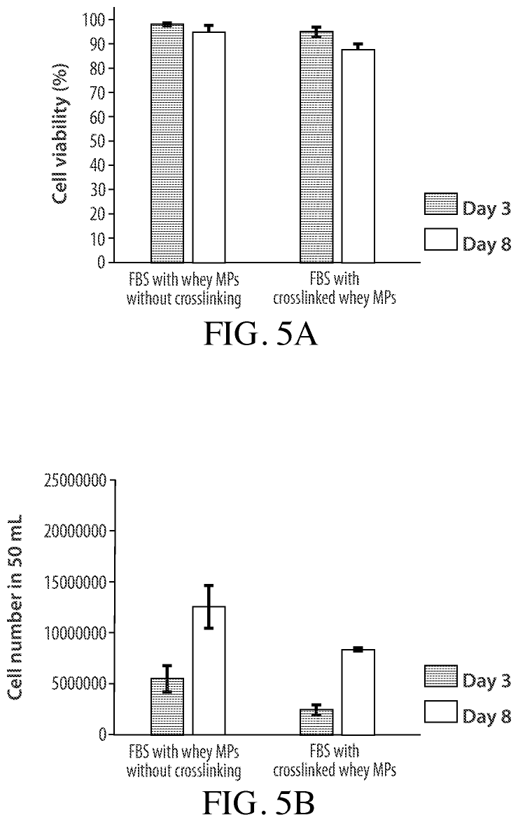

Bovine muscle cells adhered well to both crosslinked and non-crosslinked whey microparticles (FIG. 3A, FIG. 3D). Nearly all cultured cells were viable (i.e., alive) following 3 days of culture (FIG. 3A vs FIG. 3B and FIG. 3D vs FIG. 3E), under the conditions employed in the current example. High magnification images of representative cells are shown in FIG. 4. Representative photomicrographs were subsequently quantified by comparing the ratio of living cells to dead cells. The results showed that there was no difference is cell viability after 3 day or 8 days of culture, regardless of the type of microparticle (FIG. 5A).

In an additional experiment, the cells were dissociated from the microcarrier using standard protocols (e.g., trypsinization) at days 3 and 8 to determine if the cells proliferated on the crosslinked and/or non-crosslinked whey microparticles. The cell number was then quantified using standard techniques (e.g., flow cytometry, optical counting, etc.). The results showed that the cell number more than doubled between days 3 and 8 for cells grown on both crosslinked whey microparticles and non-crosslinked whey microparticles (FIG. 5B).

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teaching of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an.” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one.” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising.” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article comprising:
a whey protein; and
a coating at least partially surrounding the whey protein.

2. The article of claim 1, wherein the article further comprises a plurality of microparticles comprising the whey protein.

3. The article of claim 2, wherein the plurality of microparticles comprising the whey protein have a zeta potential less than 1.

4. The article of claim 2, wherein the plurality of microparticles comprising the whey protein have a zeta potential greater than 1.

5. The article of any one of claims 2-4, wherein the whey protein is at least partially crosslinked.

6. The article of any one of claims 2-5, wherein the coating is covalently attached to the plurality of microparticles.

7. The article of any one of claims 2-5, wherein the coating is non-covalently attached to the plurality of microparticles.

8. The article of any one of claims 2-7, wherein the plurality of microparticles has a mean diameter of between 10 microns and 100 microns.

9. The article of any one of claims 2-8, wherein the plurality of microparticles has a mean surface area of between 100 micron² and 2500 microns².

10. The article of any one of claims 2-9, wherein the plurality of microparticles has a mean aspect ratio of between 1:1 to 0.1:1.

11. The article of any one of claims 2-10, wherein at least some of the plurality of microparticles are edible.

12. The article of any one of claims 1-11, wherein the coating comprises a cell-binding ligand.

13. The article of claim 12, wherein the cell-binding ligand comprises RGD.

14. The article of any one of claim 12 or 13, wherein the cell-binding ligand comprises RGDS.

15. The article of any one of claims 1-14, wherein the coating comprises at least one component of an extracellular matrix product.

16. The article of claim 15, wherein the extracellular matrix product comprises laminin.

17. The article of any one of claims 1-16, wherein the article further comprises a plurality of animal cells.

18. The article of any one of claims 1-17, wherein the article further comprises a cultivated product.

19. A cultivated product, comprising:
a plurality of dairy-based microstructures comprising a whey protein; and
a plurality of animal cells cultured on the microstructures.

20. The cultivated product of claim 19, wherein the cultivated product comprises skin.

21. The cultivated product of any one of claim 19 or 20, wherein the cultivated product comprises leather.

22. The cultivated product of any one of claims 19-21, wherein the cultivated product comprises wool.

23. The cultivated product of any one of claims 19-22, wherein the cultivated product comprises an organ.

24. The cultivated product of any one of claims 19-23, wherein the cultivated product comprises a horn.

25. The cultivated product of any one of claims 19-24, wherein the cultivated product comprises milk.

26. The cultivated product of any one of claims 19-25, wherein the cultivated product comprises a cell-based meat.

27. A method, comprising culturing cells on a plurality of dairy-based microstructures comprising whey protein.

28. The method of claim 27, further comprising:
extracting the whey protein from a milk product; and
fabricating the plurality of dairy-based microstructures using the whey protein.

29. The method of any one of claim 27 or 28, comprising culturing the cells on the plurality of dairy-based microstructures in a cell culture system comprising a cell growth medium.

30. The method of claim 29, wherein the cell growth medium comprises at least one component capable of binding to an outer surface of the dairy-based microstructures.

31. The method of claim 30, wherein the at least one component enhances adhesion and/or proliferation of the cells on the outer surface of the microstructures.

32. The method of any one of claims 29-31, wherein the cell culture system is a bioreactor.

33. The method of any one of claims 29-32, wherein the cell growth medium comprises fetal bovine serum.

34. The method of claim 33, wherein the cell growth medium comprises soluble whey protein.

35. The method of any one of claims 27-34, further comprising removing the fetal bovine serum from the cell growth media.

36. The method of any one of claims 27-35, wherein the plurality of dairy-based microstructures comprises a plurality of microparticles.

37. The method of any one of claims 27-36, wherein the plurality of dairy-based microstructures comprises a coating.

38. The method of claim 37, wherein the coating imparts the plurality of dairy-based microstructures with a zeta potential less than 1.

39. The method of claim 37, wherein the coating imparts the plurality of dairy-based microstructures with a zeta potential greater than 1.

40. The method of any one of claims 27-39, wherein the cells are selected from the group consisting of myoblasts, fibroblasts, adipocytes, vascular cells, osteoblasts, tenocytes, epithelial cells, mammalian glands and neural cells.

41. The method of any one of claims 27-40, wherein the plurality of cells comprises fibroblasts.

42. The method of any one of claims 27-41, wherein the plurality of cells comprises adipocytes.

43. The method of any one of claims 27-42, wherein the plurality of cells comprises vascular cells.

44. The method of any one of claims 27-43, wherein the plurality of cells comprises osteoblasts.

45. The method of any one of claims 27-44, wherein the plurality of cells comprises tenocytes.

46. The method of any one of claims 27-45, wherein the plurality of cells comprises epithelial cells.

47. The method of any one of claims 27-46, wherein the plurality of cells comprises mammalian gland cells.

48. The method of any one of claims 27-47, wherein the plurality of cells comprises neural cells.

49. The method of any one of claims 27-48, wherein the plurality of cells comprises embryonic stem cells.

50. The method of any one of claims 27-49, wherein the plurality of cells comprises induced pluripotent stem cells.

51. The method of any one of claims 27-50, wherein the plurality of cells comprises adult stem cells.

52. A method, comprising:
obtaining milk from a milk-producing animal;
extracting whey protein from the milk; and
fabricating a plurality of microstructures from the whey protein.

53. The method of claim 52, wherein obtaining milk from a milk-producing animal comprises performing a plurality of milkings on a nursing milk-producing animal.

54. The method of claim 52, wherein obtaining milk from a milk-producing animal comprises performing a plurality of milkings on a pregnant milk-producing animal.

55. The method of any one of claim 53 or 54, wherein obtaining milk from a milk-producing animal comprises performing a plurality of milkings on an artificially induced milk-producing animal.

56. The method of any one of claims 53-55, wherein the plurality of milkings produces a volume of between 9,000 and 13,000 mL of milk per kg of weight of the milk producing animal.

57. The method of any one of claims 53-56, wherein the plurality of milkings produces between 50 mg and 200 mg of whey protein/mL of milk.

58. The method of any one of claims 52-57, wherein fabricating the plurality of microstructures comprises preparing a whey protein solution.

59. The method of any one of claims 52-58, wherein fabricating the plurality of microstructures comprises adding a salt solution to the whey protein solution to induce the whey proteins to fabricate the plurality of microparticles.

60. The method of any one of claims 52-59, wherein fabricating the plurality of microstructures comprises adding a crosslinking agent to the whey protein solution to induce the whey proteins to fabricate the plurality of microparticles.

61. The method of claim 60, wherein the crosslinking agent comprises transglutaminase.

62. The method of any one of claims 52-61, wherein fabricating the plurality of microstructures comprises dispersing the whey protein solution in a non-aqueous solution comprising at least one emulsifying agent to fabricate the plurality of microdroplets.

63. The method of any one of claims 52-62, wherein fabricating the plurality of protein microstructures comprises heating the whey protein solution to between 50° C. and 100° C. to fabricate the plurality of microparticles.

64. The method of any one of claims 52-63, further comprising adding the plurality of microstructures to a cell culture system comprising a plurality of cells and a cell growth medium.

65. The method of any one of claims 52-64, wherein the plurality of cells comprises fibroblasts.

66. The method of any one of claims 52-65, wherein the plurality of cells comprises adipocytes.

67. The method of any one of claims 52-66, wherein the plurality of cells comprises vascular cells.

68. The method of any one of claims 52-67, wherein the plurality of cells comprises osteoblasts.

69. The method of any one of claims 52-68, wherein the plurality of cells comprises tenocytes.

70. The method of any one of claims 52-69, wherein the plurality of cells comprises epithelial cells.

71. The method of any one of claims 52-70, wherein the plurality of cells comprises mammalian gland cells.

72. The method of any one of claims 52-71, wherein the plurality of cells comprises neural cells.

73. The method of any one of claims 52-72, wherein the plurality of cells comprises embryonic stem cells.

74. The method of any one of claims 52-73, wherein the plurality of cells comprises induced pluripotent stem cells.

75. The method of any one of claims 52-74, wherein the plurality of cells comprises adult stem cells.

76. The method of any one of claims 52-75, wherein the plurality of cells comprises human cells.

77. The method of any one of claims 52-76, wherein the plurality of cells comprises monkey cells.

78. The method of any one of claims 52-77, wherein the plurality of cells comprises cow cells.

79. The method of any one of claims 52-78, wherein the plurality of cells comprises sheep cells.

80. The method of any one of claims 52-79, wherein the plurality of cells comprises pig cells.

81. The method of any one of claims 52-80, wherein the plurality of cells comprises chicken cells.

82. The method of any one of claims 52-81, wherein the plurality of cells comprises goat cells.

83. The method of any one of claims 52-82, wherein the plurality of cells comprises bison cells.

84. The method of any one of claims 52-83, wherein the plurality of cells comprises elephant cells.

85. The method of any one of claims 52-84, wherein the plurality of cells comprises whale cells.

86. The method of any one of claims 52-85, wherein the plurality of cells comprises horse cells.

87. The method of any one of claims 52-86, wherein the plurality of cells comprises deer cells.

88. The method of any one of claims 52-87, wherein the plurality of cells comprises crocodile cells.

89. The method of any one of claims 52-88, wherein the plurality of cells comprises alligator cells.

90. The method of any one of claims 52-89, wherein the plurality of cells comprises camel cells.

91. The method of any one of claims 52-90, further comprising culturing the plurality of cells on the microstructures to produce a cultivated product.

92. A method, comprising:
dissolving whey protein in an aqueous solution to produce a whey protein solution; and
inducing the whey protein to form a plurality of microstructures.

93. The method of claim 92, wherein inducing the whey protein to form a plurality of microstructures comprises adding a salt solution to the whey protein solution.

94. The method of claim 93, comprising adding the salt solution such that the salt has a concentration in the whey protein solution of between 50 microM and 2.5M.

95. The method of any one of claims 92-94, wherein inducing the whey protein to form a plurality of microstructures comprises adding a crosslinking agent to the whey protein solution.

96. The method of claim 95, wherein the crosslinking agent comprises an enzyme.

97. The method of any one of claim 95 or 96, wherein the crosslinking agent comprises transglutaminase.

98. The method of any one of claims 95-97, wherein the crosslinking agent comprises disuccinimidyl suberate.

99. The method of any one of claims 95-98, wherein the crosslinking agent comprises sulfosuccinimidyl-4-(N-maleimidomethyl) cyclohexane-1-carboxylate.

100. The method of any one of claims 95-99, wherein the crosslinking agent comprises 1-ethyl-3-(3-dimethylaminopropyl) carbodiimide.

101. The method of any one of claims 92-100, further comprising culturing a plurality of cells on the plurality of microstructures.

102. The method of any one of claims 92-101, further comprising dissolving the plurality of microstructures.
